(19) United States
(12) Patent Application Publication
(10) Pub. No.: US 2004/0081323 A1
(43) Pub. Date: Apr. 29, 2004

(54) Noise-suppression earphone

(76) Inventor: Charles Sung, Taipei (TW)

Correspondence Address: Charles Sung, PO Box 82-144, Taipei, TW

(21) Appl. No.: 10/281,168

(22) Filed: Oct. 28, 2002

(51) Int. Cl. A61F 11/06; G10K 11/16; H03B 29/00; H04R 1/10

(52) U.S. Cl. 381/71.6; 381/72; 381/74

(57) ABSTRACT

A noise suppression earphone is constructed to include a microphone adapted to receive environmental noises, and a circuit board connected between the microphone and the voice output speaker of the earphone and adapted to set off external noises, the circuit board having a music band pass circuit, which removes high frequency and low frequency from received external noises, and a 180 deg. reverse amplifier, which reverses the waveform of input sound signal from the music band pass circuit.

### BACKGROUND OF THE INVENTION

1. Field of the Invention

The present invention relates to earphones and, more specifically, to a pair of noise-suppression earphone.

2. Description of the Related Art

Following fast development of living standards and high-tech, people care more about the quality when buying things or enjoying an entertainment. For example, in order to enjoy the performance a high quality video system, people may build up a studio in the house. However, it is quite expensive to build such a studio. Further, the studio is immovable. In order not to bother other people when listening to music, a pair of headphones or earphones may be used. However, conventional headphones or earphones cannot eliminate external noises. When listening to music with a pair of headphones under a noise environment, the user cannot enjoy hearing music without noises.

### SUMMARY OF THE INVENTION

The present invention has been accomplished under the circumstances in view. It is therefore the main object of the present invention to provide an earphone, which sets off external noises when listening to music. It is another object of the present invention to provide a noise suppression earphone, which is inexpensive. To achieve these and other objects of the present invention, the noise suppression earphone comprises a microphone adapted to receive environmental noises, and a circuit board connected between the microphone and the voice output speaker of the earphone and adapted to set off external noises, the circuit board having a music band pass circuit, which removes high frequency and low frequency from received external noises, and a 180 deg. reverse amplifier, which reverses the waveform of input sound signal from the music band pass circuit. Further, an on/off switch may be provided and extended to the outside of the earphone for operation by hand to switch on/off the circuit board.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 a perspective view of a pair of headphones constructed according to the preferred embodiment of the present invention.

FIG. 2 is a circuit block diagram according to the preferred embodiment of the present invention.

FIG. 3 is a detailed circuit diagram of the preferred embodiment of the present invention.

### DETAILED DESCRIPTION OF THE PREFERRED EMBODIMENT

The following descriptions are of exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

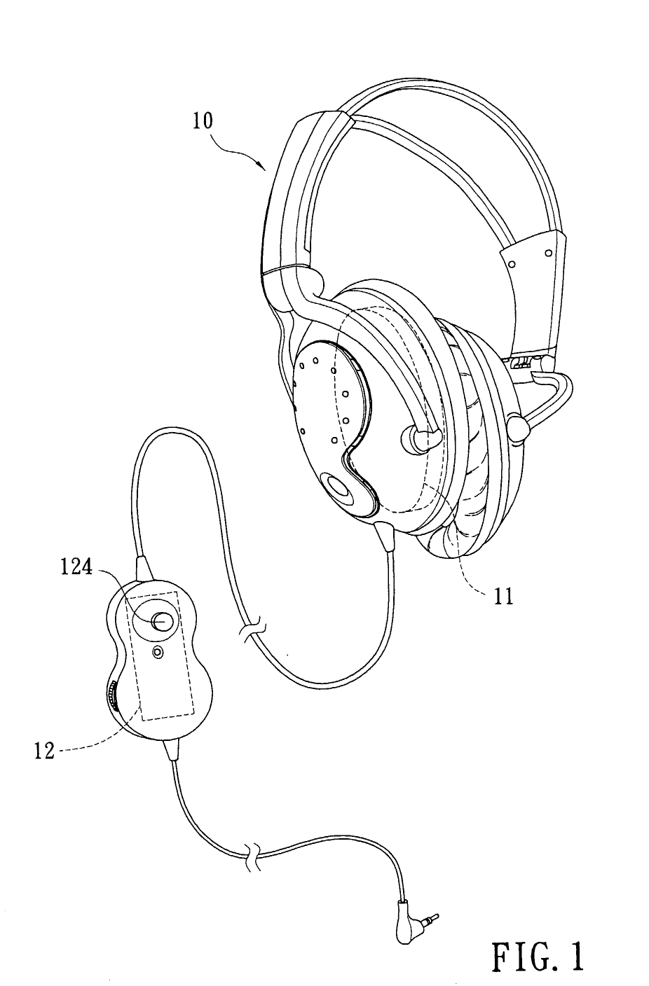

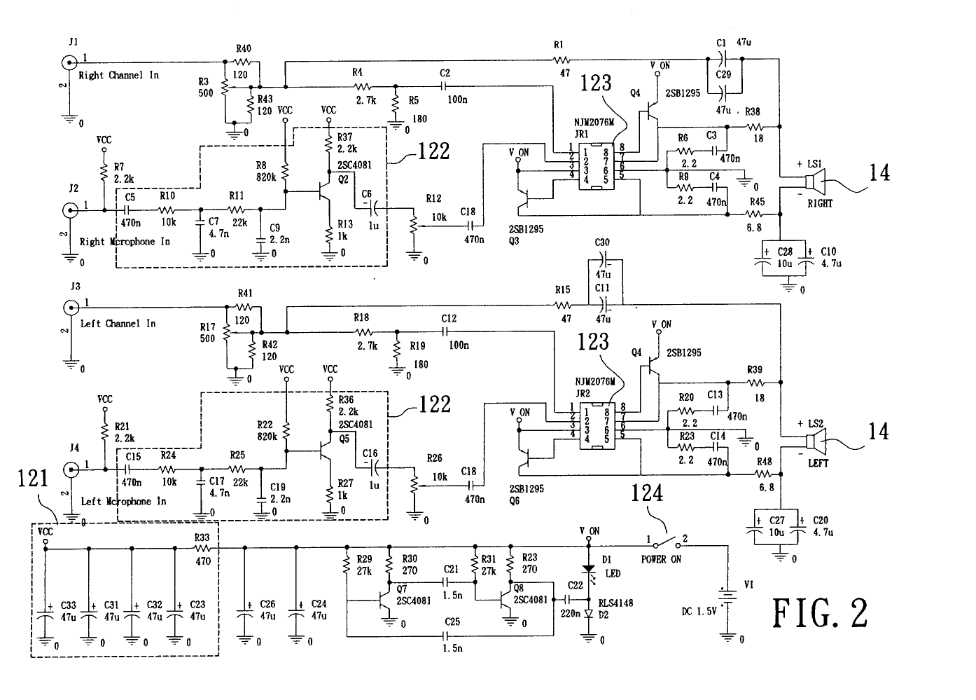

Referring to FIGS. 1 and 2, the housing 11 of each earphone unit of the headphones, referenced by 10, holds a microphone 13, which is electrically connected to the voice signal input port of a respective circuit board 12 (only one circuit board is shown in the drawings), which has its voice signal output port connected to a respective speaker 14.

Referring to FIG. 2, the aforesaid circuit board 12 comprises a music band pass circuit 121, a 180 deg. reverse amplifier 122, a diode, and a switch 124. The music band pass circuit 121 is formed of resistors, capacitors, transistors, a power amplifier 123, and a LED. When under a quite environment, the switch 124 can be switched off. When the noise suppression headphones 10 receives left channel music and right channel music from a music source under a noisy environment and the switch 124 maintained off, external noises interfere with left channel music and right channel music, and the user cannot hear the music clearly.

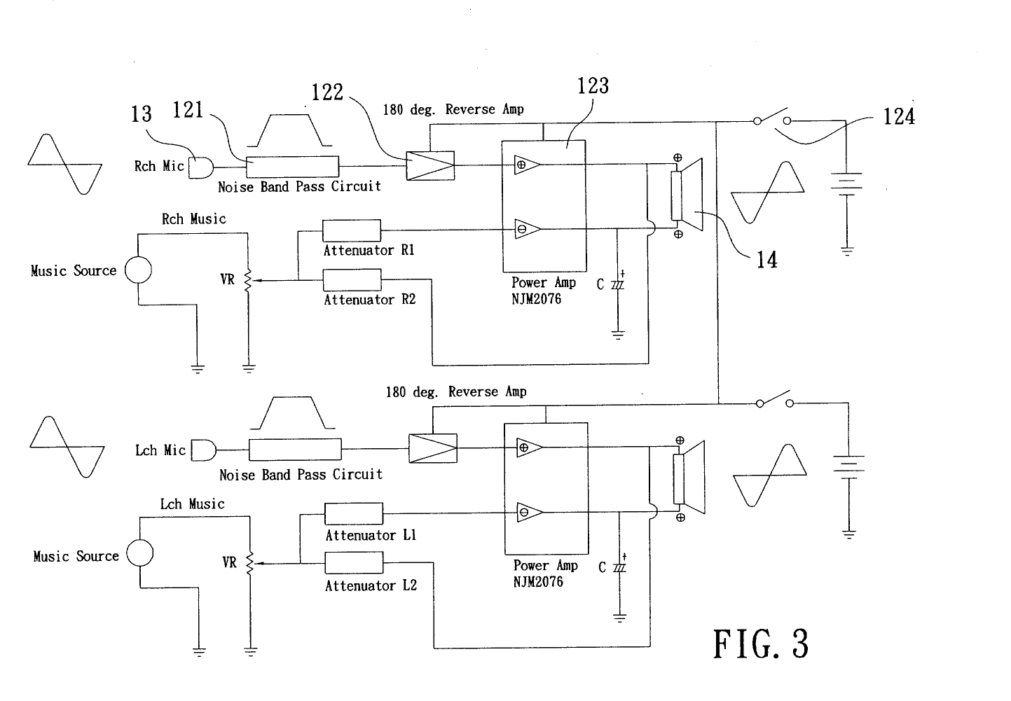

Referring to FIG. 3, when switched on the switch 124, the microphone 13 receives environmental noises, and sends received environmental noises through the music band pass circuit 121, which removes high frequency and low frequency from received environmental noises, and the reverse amplifier 122, which reverses the waveform and then inputs it with inputted music into the power amplifier 123 for amplification, for enabling external noises to be offset. Thus, only amplified music signal is outputted through the speaker 14.

As indicated above, the invention has numerous advantages as follows:

1. The microphone and the circuit board can be installed in a conventional earphone, forming a noise suppression earphone.

2. The earphone can be used anywhere under any noisy environments.

3. The use of the noise suppression earphone eliminates the necessity of building an expensive studio in the house.

A prototype of noise suppression earphone has been constructed with the features of FIGS. 1-3. The noise suppression earphone functions smoothly to provide all of the features discussed earlier.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A noise suppression earphone comprising a microphone adapted to receive external noises, and a circuit board connected between said microphone and a voice output speaker of the noise suppression earphone and adapted to set off external noises received by said microphone, said circuit board comprising a music band pass circuit, which removes high frequency and low frequency from received external noises, and a 180 deg. reverse amplifier, which reverses the waveform of input sound signal from said music band pass circuit.

2. The noise suppression earphone as claimed in claim 1, wherein said music band pass circuit is formed of resistors, capacitors, transistors, a power amplifier, and a LED.

3. The noise suppression earphone as claimed in claim 1, wherein said circuit board further comprises an on/off switch extended to the outside of the earphone for switching on/off said circuit board.

4. The noise suppression earphone as claimed in claim 1, which has a housing, forming one earphone unit of a headphones.
